(19) United States
(12) Patent Application Publication
(10) Pub. No.: US 2016/0262917 A1
(43) Pub. Date: Sep. 15, 2016

(54) BIOERODIBLE POLYMERIC MEDICAL DEVICE HAVING PHOTO ACTIVE GROUPS

(71) Applicant: BOSTON SCIENTIFIC SCIMED, INC., Maple Grove, MN (US)

(72) Inventors: JOHN J. CHEN, Plymouth, MN (US); STEVEN L. KANGAS, Woodbury, MN (US); YEN-LANE CHEN, New Brighton, MN (US); HONGXIA ZENG, Maple Grove, MN (US); RAGHAV GOEL, New Delhi (IN)

(21) Appl. No.: 15/066,649

(22) Filed: Mar. 10, 2016

Related U.S. Application Data
(60) Provisional application No. 62/131,425, filed on Mar. 11, 2015.

(51) Int. Cl. A61F 2/90; A61L 31/14; B29C 35/08; A61L 2/00; B29C 47/00; B29C 35/02; A61L 31/06; A61F 2/04

(52) U.S. Cl. CPC A61F 2/90; A61L 31/06; A61L 31/148; A61F 2/04; A61L 2/007; B29C 47/0004; B29C 47/0023; B29C 47/0066; B29C 35/0266; B29C 35/0866; B29C 35/0805; A61F 2210/0076; A61F 2230/0069; A61F 2240/001; B29C 2035/0877; B29C 2035/0827; B29L 2023/22

(57) ABSTRACT

A medical device includes a tubular network of struts including a bioerodible polymer. The tubular network can include a plurality of bands and a plurality of connector struts. Each band can include a plurality of peaks between adjacent band struts. Each band can be connected to one or more adjacent bands by at least two connector struts at a plurality of connector intersections. The bioerodible polymer can include a plurality of polymer chains functionalized with a photo active group.

### CROSS-REFERENCE TO RELATED APPLICATIONS

This application claims priority under 35 U.S.C. §119 to U.S. Provisional Application Ser. No. 62/131,425, filed Mar. 11, 2015, the entirety of which is incorporated herein by reference.

### TECHNICAL FIELD

This invention relates to bioerodible polymeric medical devices such as stents that include a photo active group in the bioerodible polymer.

### BACKGROUND

Medical devices, such as stents, can be implanted to permanently or temporarily support or replace diseased tissue. For example, stents can be cylindrically shaped devices designed to hold open and sometimes expand a segment of a blood vessel or other anatomical lumen such as urinary tracts and bile ducts. In some cases, it is desirable for the medical device to be absorbed after surrounding tissues have healed. Recent evidence suggests that the continued presence of a metallic stent in the coronary artery may later alter blood flow dynamics, restrict normal vascular response, and potentially limit maximal vasodilation. Permanent metallic stenting may also prevent surgical revascularization, inhibit late lumen gain, and limit noninvasive imaging of the coronary arteries using computed tomography (CT) and magnetic resonance imaging (MRI), due in part to the metallic stents possibly causing a blooming effect with these imaging modalities, which can make interpretation more difficult.

Bioerodible polymeric medical devices, such as stents, have been proposed to overcome the difficulties associated with permanent metallic devices, but pose their own challenges. For example, in the case of a balloon-expandable stent, which is typically crimped around a balloon of a balloon catheter for delivery and plastically expanded at an anatomical location by inflating the balloon, the crimping and expansion can damage the polymer of the stent, particularly at locations where the stent is designed to plastically deform. This damage can result in a premature failure of the bioerodible polymeric medical device. In addition, the mechanical properties of the polymer can be negatively impacted by the processes used to manufacture the stent. For example, the process to make a polymer stent usually involves a 1^{st} extrusion of the polymer into a tube. Melt extrusion of biodegradable polymers such as PLLA results in a significant reduction of the polymer molecular weight which leads to a more brittle polymer. In addition, e-beam sterilization of PLLA leads to a further reduction in polymer molecular weight. One way to overcome this loss in properties is to start with a polymer resin with a higher molecular weight. However, there is a limit to the molecular weight that can be extruded due to viscosity effects and the fact that degradation during extrusion can increase with molecular weight. A reduction in key mechanical properties (leading to reduced strain at break) can result in strut fracture in the high strain regions of the stent. The selection of more resilient bioerodible polymeric materials for the stent, however, can undermine the other mechanical properties desirable in a stent. Attempts to provide different materials in different locations of a stent, however, can add cost and complexity to a stent manufacturing process.

### SUMMARY

Medical devices provided herein can include a bioerodible polymer having increased chain extension or crosslinking in select areas or throughout the entire stent to improve the resistance to damage caused by deformation of the medical device during use. For example, medical devices provided herein can be stents designed to plastically deform at one or more peaks and/or one or more connector locations.

In Example 1, a medical device includes a tubular network of struts including a bioerodible polymer. The tubular network can include a plurality of bands and a plurality of connector struts. Each band can include a plurality of peaks between adjacent band struts and be connected to one or more adjacent bands by at least two connector struts at a plurality of connector intersections. The bioerodible polymer can include a plurality of polymer chains functionalized with a photo active group.

In Example 2, the medical device of Example 1, wherein the photo active group is a cinnamate functional group.

In Example 3, the medical device of Example 2, wherein a plurality of the polymer chains each include two or more terminal cinnamate functional groups.

In Example 4, the medical device of one of Examples 1-3, wherein the polymer chains are heat stable such that exposure to a temperature of 215° C. for 20 minutes does not cause more than 5% of the polymer chains to crosslink or chain extend.

In Example 5, the medical device of one of Examples 1-4, wherein the bioerodible polymer includes a polymer selected from the group consisting of PLGA, PDLA, PLLA, PCL, PHBV, POE, PEO/PBTP, one or more polyamides, one or more polyanhides, and a combination thereof.

In Example 6, the medical device of Example 5, wherein the bioerodible polymer includes a biodegradable polyester functionalized with cinnamate functional groups.

In Example 7, the medical device of one of Examples 1-6, wherein the bioerodible polymer has a number molecular weight of at least 30,000 and a Tg of at least 40° C.

In Example 8, the medical device of one of Examples 1-7, wherein each peak includes crosslinked or chain extended bioerodible polymer and each band strut between the peaks includes bioerodible polymer that is less crosslinked or chain extended than the peaks.

In Example 9, the medical device of one of Examples 1-8, wherein the tubular network includes greater crosslinking or chain extension between polymer chains in one or more peaks and/or one or more connector intersections as compared to one or more mid sections of adjacent struts.

In Example 10, a method of forming the medical device of one of Examples 1-9 includes providing a tubular network of struts including a bioerodible polymer. The tubular network can include a plurality of bands and a plurality of connector struts, each band including a plurality of peaks between adjacent band struts and being connected to one or more adjacent bands by at least two connector struts at a plurality of connector intersections. The bioerodible polymer can include a plurality of polymer chains functionalized with a photo active group. The method can also include selectively focusing light onto one or more peaks, one or more connector intersections, or a combination thereof. The light can have a wavelength adapted to trigger the crosslinking or chain extension of the bioerodible polymer in the one or more peaks, one or more connector intersections, or a combination thereof.

In Example 11, the method of Example 10, wherein the light is UV light having a predetermined wavelength.

In Example 12, the method of Example 10 or Example 11, further including forming the tubular network by melt extruding the bioerodible polymer into a tube and cutting fenestrations in the tube to define the bands and the connectors, wherein the melt extruding of the bioerodible polymer does not trigger the decomposition of the photo active group.

In Example 13, the method of one of Examples 10-12, further including sterilizing the tubular network with e-beam radiation, wherein the e-beam radiation does not trigger the decomposition of the photo active group.

In Example 14, the method of one of Examples 10-13, further including functionalizing a bioerodible polymer with one or more functional groups to form the bioerodible polymer.

In Example 15, the method of one of Examples 10-14, wherein the bioerodible polymer includes a biodegradable polyester functionalized with cinnamate functional groups.

In Example 16, a medical device includes a tubular network of struts including a bioerodible polymer. The bioerodible polymer can include a plurality of polymer chains functionalized with a photo active group the tubular network including a plurality of bands and a plurality of connector struts. Each band can include a plurality of peaks between adjacent band struts and be connected to one or more adjacent bands by at least two connector struts at a plurality of connector intersections. The tubular network can include greater crosslinking or chain extension between polymer chains in one or more peaks, one or more connector intersections, or a combination thereof as compared to one or more midsections of adjacent struts.

In Example 17, the medical device of Example 16, wherein the photo active group is a cinnamate functional group.

In Example 18, the medical device of Example 17, wherein a plurality of the polymer chains each include two or more terminal cinnamate functional groups.

In Example 19, the medical device of Example 16, wherein the polymer chains are heat stable such that exposure to a temperature of 215° C. for 20 minutes does not cause more than 5% of the polymer chains to crosslink or chain extend.

In Example 20, the medical device of Example 16, wherein the bioerodible polymer includes a polymer selected from the group consisting of PLGA, PDLA, PLLA, PCL, PHBV, POE, PEO/PBTP, one or more polyamides, one or more polyanhides, and a combination thereof.

In Example 21, the medical device of Example 20, wherein the bioerodible polymer consists essentially of PLLA functionalized with cinnamate functional groups.

In Example 22, the medical device of Example 16, wherein the bioerodible polymer has a number molecular weight of at least 30,000 and a Tg of at least 40° C.

In Example 23, the medical device of Example 16, wherein each peak includes crosslinked or chain extended bioerodible polymer and each band strut between the peaks includes bioerodible polymer that is less crosslinked or chain extended than the peaks.

In Example 24, the medical device of Example 16, wherein the medical device is a stent.

In Example 25, a method of forming a medical device including focusing light onto one or more predetermined locations of a tubular network, the tubular network including a bioerodible polymer, the bioerodible polymer including a plurality of polymer chains functionalized with a photo active group, the tubular network including a plurality of bands and a plurality of connector struts, each band including a plurality of peaks between adjacent band struts and being connected to one or more adjacent bands by at least two connector struts at a plurality of connector intersections, the light having a wavelength adapted to trigger the crosslinking or chain extension of the bioerodible polymer in the one or more predetermined locations.

In Example 26, the method of Example 25, wherein the light is UV light have a predetermined wavelength.

In Example 27, the method of Example 25, further including forming the tubular network by melt extruding the bioerodible polymer into a tube and cutting fenestrations in the tube to define the bands and the connectors, wherein the melt extruding of the bioerodible polymer does not trigger the decomposition of the photo active group.

In Example 28, the method of Example 25, further including sterilizing the tubular network with e-beam radiation, wherein the e-beam radiation does not trigger the decomposition of the photo active group.

In Example 29, the method of Example 25, further including functionalizing a bioerodible polymer with one or more functional groups to form the bioerodible polymer.

In Example 30, the method of Example 25, wherein the photo active group is a cinnamate functional group.

In Example 31, the method of Example 25, wherein a plurality of the polymer chains each include two or more terminal cinnamate functional groups.

In Example 32, a stent includes a tubular network of struts including a PLLA chains functionalized with one or more cinnamate functional groups.

In Example 33, the stent of Example 32, wherein a plurality of PLLA chains each include two terminal cinnamate functional groups.

In Example 34, the stent of Example 32, wherein the PLLA chains are heat stable such that exposure to a temperature of 215° C. for 20 minutes does not cause more than 5% of the polymer chains to crosslink or chain extend or chain extend.

In Example 35, the stent of Example 32, wherein the functionalized PLLA chains have a number molecular weight of at least 30,000 and a Tg of at least 40° C.

Medical devices provided herein can include polymer chains functionalized with photo active functional groups. Methods provided herein include the focusing of light (e.g., UV light of a predetermined wavelength) onto select locations of a tubular network having photo active functional groups in order to selectively chain extend or crosslink polymer chains in the select locations or to chain extend or crosslink the entire stent. Bioerodible polymers containing photo active groups provided herein can be stable during processing (e.g., melt extruding, e-beam sterilization) such that the photo active group of the bioerodible polymer does not unintentionally crosslink or chain extend at undesired locations. In some cases, the photo active group of one polymer chain can be adapted to covalently couple with another photo active group on another polymer chain at a predetermined wavelength. In some cases, a medical device provided herein can be included in a package that blocks the one or more predetermined initiating wavelengths for the photo active group. An advantage of the medical devices and methods provided herein is that the medical devices are resistant to unintended crosslinking or chain extension in the bioerodible polymer during production of the tubular network, in areas surrounding preselected locations when cross-linking preselected locations, during sterilization, and during packaging, storage, and shipment.

In some cases, the tubular network can include greater crosslinking or chain extension between polymer chains in one or more peaks and/or one or more connector intersections as compared to one or more mid sections of adjacent struts. In some cases, the photo active group is a cinnamate functional group. In some cases, the photo active group is a derivative of a cinnamate functional group, for example, 1,3-diphenyl-2-propen-1-on (e.g., Chalkon), cinnamylacylic acid, 4-methylcoumarin, and ortho-substituted cinnamates. In some cases, the photo active group can include a functional group that includes, but is not limited to, alkyne, anthracene, benzothiophene dioxide, chalcone, cinnamate, cournarin, dibenzazepine, diphenylcyclopropene-carboxylate,episulfide,maleimide, stilbazole, stilbene, styrene, 1,2,3-thiadiazole and thymine. In some cases, a plurality of the polymer chains can each include two or more terminal cinnamate functional groups. In some cases, the polymer chains are heat stable such that exposure to a temperature of 215° C. for 20 minutes does not cause more than 5% of the polymer chains to crosslink or chain extend. In some cases, the bioerodible polymer can be a polymer selected from the group consisting of PLGA, PDLA, PLLA, PCL, PHBV, POE, PEO/PBTP, one or more polyamides, one or more polyanhides, and a combination thereof. In some cases, the polymer can be PLLA. In some cases, the bioerodible polymer can include a biodegradable polyester functionalized with cinnamate functional groups. In some cases, the bioerodible polymer has a number molecular weight of at least 30,000 and a Tg of at least 40° C. In some cases, each peak includes crosslinked or chain extended bioerodible polymer and each band strut between the peaks includes bioerodible polymer that is less crosslinked or chain extended than the peaks.

In some aspects, a method of forming a medical device includes providing a tubular network of struts including a bioerodible polymer and selectively focusing light onto one or more peaks, one or more connector intersections, or a combination thereof. The tubular network can include a plurality of bands and a plurality of connector struts. Each band can include a plurality of peaks between adjacent band struts and being connected to one or more adjacent bands by at least two connector struts at a plurality of connector intersections. The bioerodible polymer can include a plurality of polymer chains functionalized with a photo active group. The light can have a wavelength adapted to trigger the crosslinking or chain extension of the bioerodible polymer in the one or more peaks, one or more connector intersections, or a combination thereof. In some cases, the light is UV light having a predetermined wavelength. In some cases, the method can include forming the tubular network by melt extruding the bioerodible polymer into a tube and cutting fenestrations in the tube to define the bands and the connectors. In some cases, the melt extruding of the bioerodible polymer does not trigger the decomposition of the photo active group. In some cases, the method can include sterilizing the tubular network with e-beam radiation. The e-beam radiation can be selected such that it does not trigger the decomposition of the photo active group. In some cases, the method can further include functionalizing a bioerodible polymer with one or more functional groups to form the bioerodible polymer. In some cases, the bioerodible polymer includes a biodegradable polyester functionalized with cinnamate functional groups. In some cases, the bioerodible polymer has a number molecular weight of at least 30,000 and a Tg of at least 40° C.

Medical devices provided herein can include any suitable bioerodible polymer functionalized with a photo active group. In some cases, the bioerodible polymer can be selected from the group consisting of PLGA, PDLA, PLLA, PCL, PHBV, POE, PEO/PBTP, one or more polyamides, one or more polyanhydrides, and a combination thereof functionalized with one or more photo active groups. In some cases, the bioerodible polymer can be a bioerodible polyester functionalized with one or more photo active groups. In some cases, medical devices provided herein can include cinnamate functionalized PLLA having a number molecular weight of at least 30,000. In some cases, medical devices provided herein can include cinnamate functionalized PLLA having a Tg of at least 40° C. In some cases, stents provided herein can include cinnamate functionalized PLLA having a number molecular weight of at least 30,000 and a Tg of at least 40° C. In some cases, medical devices provided herein can be stents. Stents provided herein provide suitable ductility to allow for crimping, expansion, and cyclic loading. Stents provided herein can provide improved longitudinal flexibility to allow the stent to be maneuvered through a tortuous vascular path and to enable it to conform to a deployment site that may not be linear or may be subject to flexure.

### DESCRIPTION OF DRAWINGS

FIG. 1 is a perspective view of an exemplary medical device provided herein.

FIG. 2 depict how an exemplary bioerodible polymeric tubular network can be damaged due to crimping and expansion.

FIGS. 3A and 3B depict how another bioerodible polymeric tubular network can be damaged due to crimping and expansion.

FIG. 4 depicts how a medical device provided herein can be treated with light to crosslink or chain extend select locations.

FIG. 5A illustrates the structure of cinnamate. FIG. 5B depicts how terminal cinnamate functional groups can crosslink or chain extend bioerodible polymer chains

FIG. 6A depicts how a carboxylic acid end group of PLLA can be converted to a hydroxyl end group by a reaction with a diol. FIG. 6B depicts endcapping of a PLLA diol with cinnamate groups. FIG. 6C depicts how UV light can cause difunctional cinnamate PLLA to undergo chain extension resulting in an increase in number molecular weight.

FIG. 7A depicts how a carboxylic acid end group of PLLA can be converted to multiple hydroxyl end groups by reaction with a triol. FIG. 7B depicts endcapping of a PLLA triol with cinnamate groups. FIG. 7C depicts how UV light can cause trifunctional cinnamate functionalized PLLA to crosslink or chain extend to create a network of crosslinked or chain extended polymer chains.

Like reference symbols in the various drawings indicate like elements.

### DETAILED DESCRIPTION

Medical devices and methods provided herein include a bioerodible polymer having polymer chains functionalized with a photo active group. A photo active functional group provided herein can be bound to any bioerodible polymer suitable for use in a medical device. In some cases, the photo active functional groups can be covalently bound to a polymer chain of the bioerodible polymer. In methods provided herein, light can be selectively directed towards select portions of a photo active functionalized bioerodible polymer to preferentially chain extend or crosslink polymer chains in the select portions. As will be discussed below, photo active functionalized bioerodible polymers provided herein can be thermally stable and minimize unintentional crosslinking or chain extension during formation of the medical device or during sterilization, packaging, shipping and/or storage of the medical device.

### Bioerodible Polymer

Medical devices provided herein include any suitable photo active functionalized bioerodible polymer. In some cases, the photo active functionalized bioerodible polymer can be selected from the group consisting of poly(lactide-co-glycolide) (PLGA), poly(D,L-lactic acid) (PDLA), poly(L-lactic acid) (PLLA), poly(caprolactone) (PCL), polyhydroxy-butyrate/-valerate copolymer (PHBV), polyorthoester (POE), polyethyleneoxide/polybutylene terephthalate copolymer (PEO/PBTP), one or more polyamides (such as Nylon 66 and polycaprolactam), one or more polyanhidride, and a combination thereof each functionalized with one or more photo active groups. In some cases, the photo active functionalized bioerodible polymer can be a bioerodible polyester. Suitable bioerodible polyesters include, but are not limited to, poly(L-lactide) (PLLA), poly(DL-lactide) (PDLLA), polyglycolide (PGA), xzpoly(glycolide-co-lactide) (PLGA), poly(L-lactide-co-caprolactone) (PLLA-co-PCL), poly(L-lactide-co-trymethylene carbonate) (PLLA-co-PTMC), and poly(L-lactide-co-DL-lactide) (PLLA-co-PDLLA).

For example, the photo active functionalized bioerodible polymer can include a photo active group and PLLA. The application of light (e.g., UV laser light) can cause the photo active group to decompose into a free radical. The free radical can then react with a portion of PLLA to create a reactive PLLA having a free radical, which can then react with other reactive PLLA chains to create a cross-linked network of PLLA.

In some cases, the photo active functionalized PLLA can have a number molecular weight of at least 30,000. In some cases, the photo active functionalized PLLA can have a Tg of at least 40° C. In some cases, the photo active functionalized PLLA can have a number molecular weight of at least 30,000 and a Tg of at least 40° C. Additional examples of polymers that may be functionalized with a photo active group and used in a medical device provided herein include, but are not limited to, poly(N-acetylglucosamine) (Chitin), Chitosan, poly(hydroxyvalerate), poly(hydroxybutyrate), poly(hydroxybutyrate-co-valerate), polyanhydride, poly(glycolic acid), poly(glycolide), poly(L-lactide), poly(D,L-lactic acid), poly(D,L-lactide), polyester amide, poly(glycolic acid-co-trimethylene carbonate), co-poly(ether-esters) (e.g. PEO/PLA), polyphosphazenes, and biomolecules (such as fibrin, fibrinogen, cellulose, starch, collagen and hyaluronic acid). Another type of polymer based on poly(lactic acid) that can be used includes graft copolymers, and block copolymers, such as AB block-copolymers (“diblock-copolymers”) or ABA block-copolymers (“triblock-copolymers”), or mixtures thereof.

Photo active functionalized bioerodible polymers provided herein can be completely amorphous, partially crystalline, or almost completely crystalline. A partially crystalline polymer includes crystalline regions separated by amorphous regions. The crystalline regions do not necessarily have the same or similar orientation of polymer chains. However, a high degree of orientation of crystallites may be induced by applying stress to a semi-crystalline polymer. The stress may also induce orientation in the amorphous regions. An oriented amorphous region also tends to have high strength and high modulus along an axis of alignment of polymer chains. Additionally, for some polymers under some conditions, induced alignment in an amorphous polymer may be accompanied by crystallization of the amorphous polymer into an ordered structure. This is known as stress induced crystallization.

In some cases, medical devices provided herein can be fully bioerodible. In some cases, medical devices including a tubular network of struts provided herein can include a photo active functionalized bioerodible polymer that is the primary source of the radial strength of the tubular network. In some cases, a medical device provided herein is a stent. In some cases, stents provided herein are completely or primarily composed of bioerodible polymer. In some cases, bands of stents provided herein are substantially free of metallic material. In some cases, only radiopaque markers of the stent include metallic materials.

### Photo Active Group

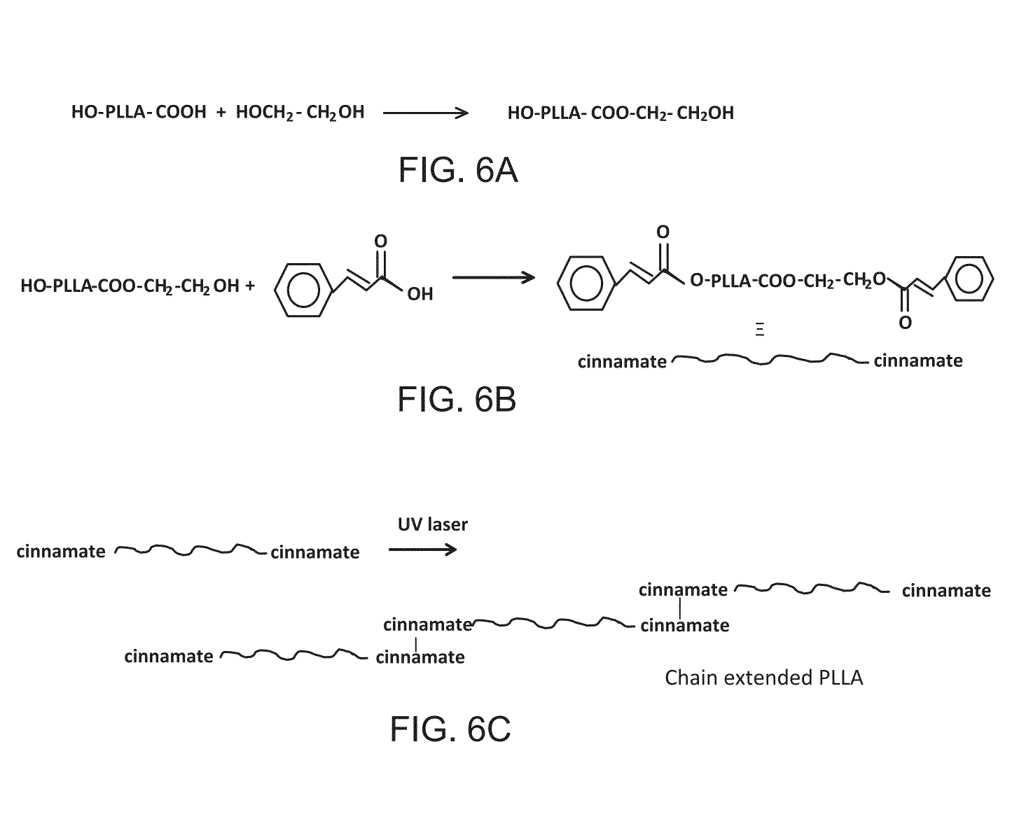

As shown in FIGS. 5A-5B and discussed below, bioerodible polymer provided herein can be functionalized with cinnamate groups, which are photo active and can couple via a cycloaddition reaction when irradiated with UV light of a wavelength of 365 nm (FIG. 5B).

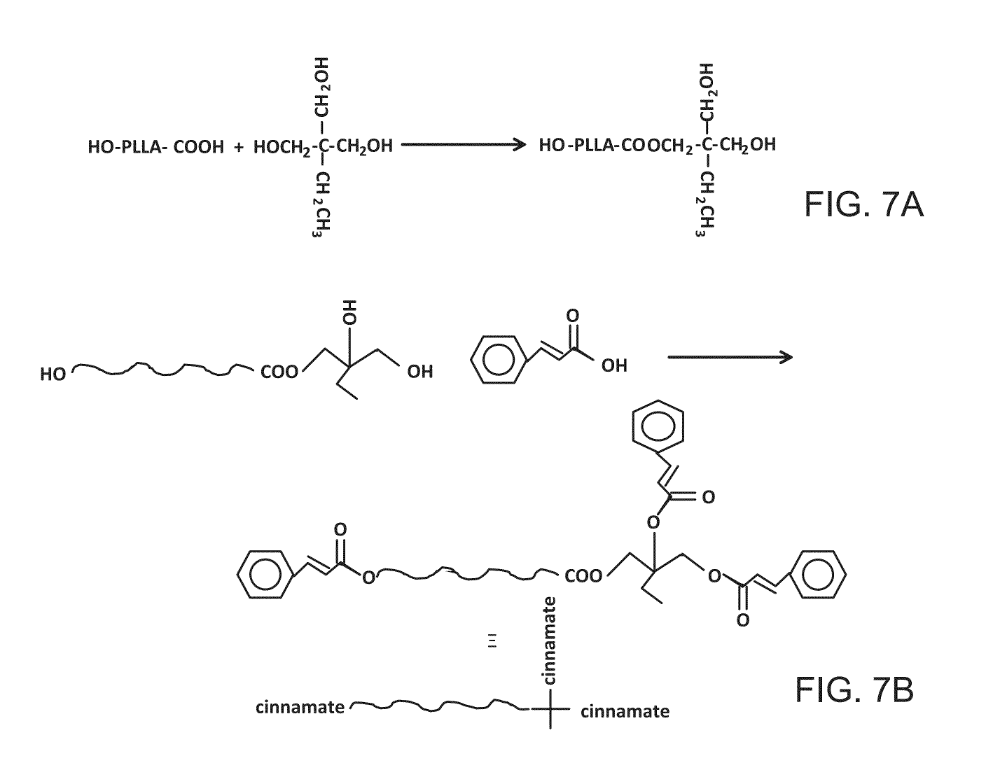

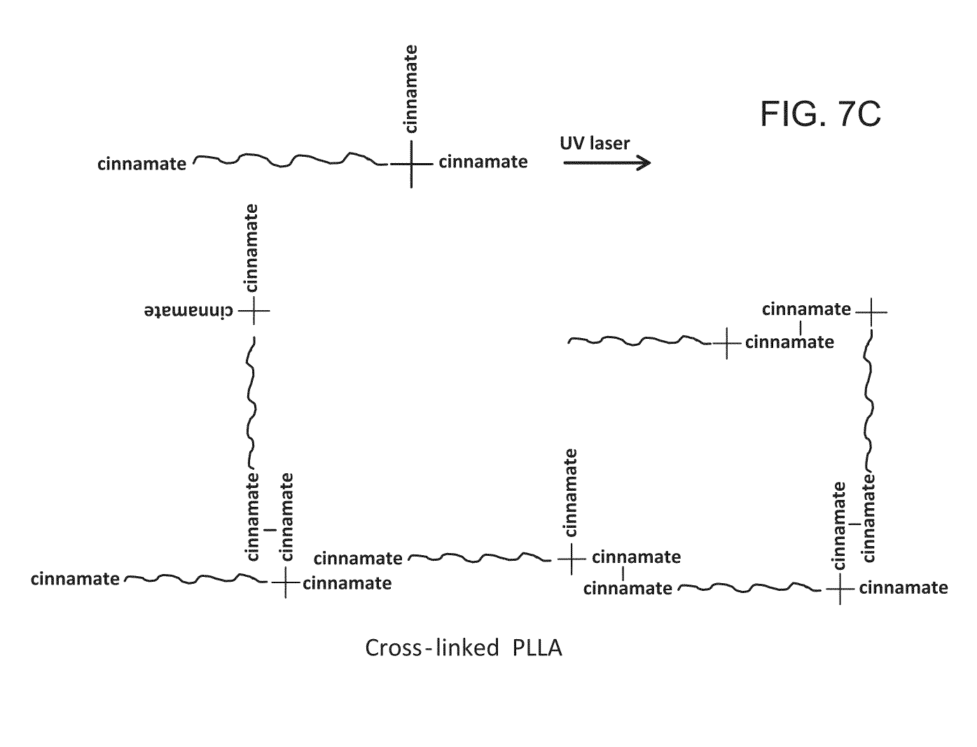

Bioerodible polymers provided herein can be functionalized with photo active groups provided herein using any suitable method. The following embodiments describe how PLLA can be functionalized with cinnamate. PLLA, when polymerized, is encapped by a hydroxyl group on one end of the polymer chain and a carboxylic acid on the other end. The acid terminated end can be converted to a hydroxyl, for example by reacting the PLLA with an excess of a diol such as an ethylene glycol. This gives a difunctional hydroxyl PLLA (FIG. 6A). Alternatively, the carboxylic acid end group can be converted into a multifunctional hydroxyl, for example, by reacting the PLLA with a triol such as a trimethylopropane. This gives a trifunctional PLLA (FIG. 7A). The resulting hydroxyl functional PLLA can be reacted with cinnamic acid under mild conditions, for example, by reacting the PLLA with the acid chloride of cinnamic acid or by using cinnamic acid and a coupling agent such as a carbodiimide. Endcapping the hydroxyl difunctional PLLA with cinnamate leads to the difunctional cinnamate (FIG. 6B). Upon exposure to UV light, the PLLA undergoes a chain extension with a resulting increase in number molecular weight (FIG. 6C). End capping a trifunctional hydroxyl PLLA with cinnamic acid results in a trifunctional cinnamate (FIG. 7B). Upon UV crosslinking or chain extending, the PLLA forms a crosslinked network (FIG. 7C).

In some cases, end groups of a bioerodible polymer (e.g., a bioerodible polyester) can be reacted to substitute the end group with a photo active functional group. In some cases, the hydroxyl end groups of a bioerodible polymer (e.g., a bioerodible polyester) can be replaced with selected photo active functional groups. In some cases, some or all of the hydroxyl end groups of a bioerodible polymer can be replaced with selected photo active end groups. In some cases, less than 10%, 10-30%, 30-70%, or greater than 70% of hydroxyl groups can be replaced by the selected photo active functional groups. In some cases, the bioerodible polymer chains of a bioerodible polymer can be linear, star, branched, or dendritic.

In some cases, the bioerodible polymer provided herein can be functionalized with a derivative of a cinnamate functional group, for example, 1,3-diphenyl-2-propen-1-on (e.g., Chalkon), cinnamylacylic acid, 4-methylcoumarin, and ortho-substituted cinnamates.

In some cases, the bioerodible polymer provided herein can be functionalized with a photo active group that includes, but is not limited to, alkyne, anthracene, benzothiophene dioxide, chalcone, cinnamate, cournarin, dibenzazepine, diphenylcyclopropene˜carboxylate,episulfide,maleimide, stilbazole, stilbene, styrene, 1,2,3-thiadiazole and thymine.

### Medical Devices

Implantable medical devices and methods of fabricating medical devices made from polymers are provided herein. In some cases, medical devices provided herein can be selectively crosslinked or chain extended, yet have improved thermal stability during one or more processing steps of a fabrication process. Such processing steps can include exposing the polymer to conditions such as elevated temperatures and radiation that can cause chemical degradation to a polymer. The methods and devices described herein are generally applicable to any implantable medical device. In particular, the methods can be applied to tubular implantable medical devices such as self-expandable stents, balloon-expandable stents, stent-grafts, and pacemaker leads. Additionally, medical devices provided herein can be made partially or completely of the photo active functionalized bioerodible polymer provided herein. For example, a device body, such as a stent scaffolding, can be made from the photo active functionalized bioerodible polymer provided herein. Producing a tubular network entirely of a single type of polymer and using light of a predetermined wavelength to selectively crosslink or chain extend the polymer can allow for a simplified yet controlled manufacturing of a tubular network having different material properties in different sections of the stent. Additionally, in some cases, a coating over a device body can also be made from the photo active functionalized bioerodible polymer provided herein.

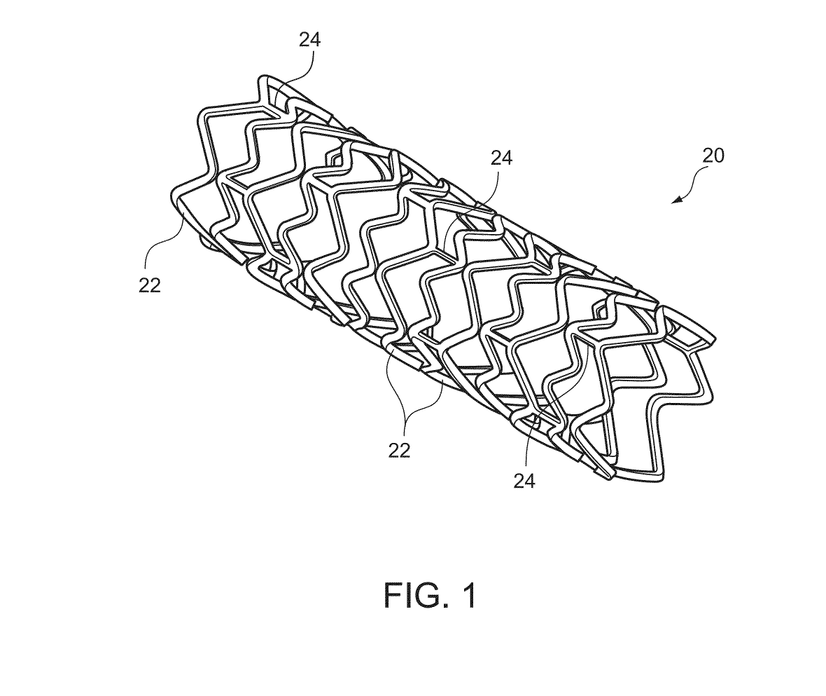

FIG. 1 depicts stent 20, an example of a stent provided herein. Stent 20 has a cylindrical shape. Stent 20 includes a plurality of bands 22. Each internal band 22 is connected to two adjacent bands by a plurality of connectors 24. Each connector 24 extends between adjacent bands. In some cases, select connectors 24 can include radiopaque markers (not shown). Stent 20 can be a self-expandable stent or a balloon-expandable stent, or part of a stent-graft.

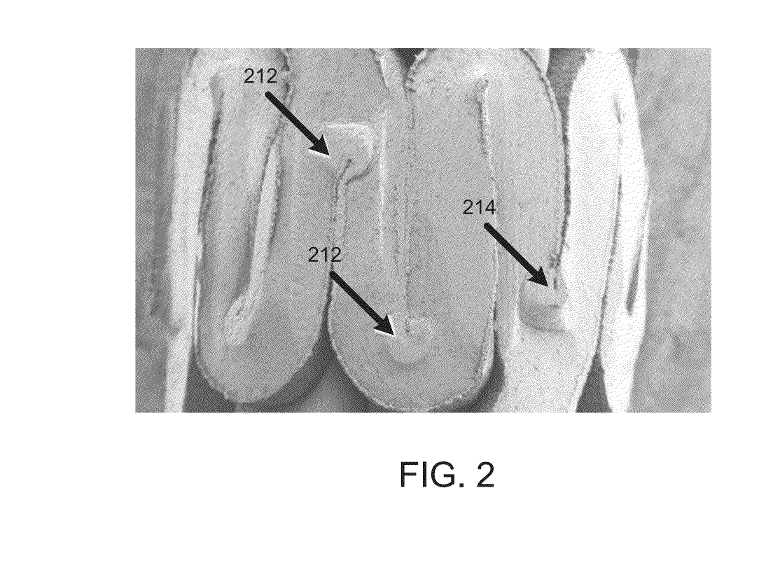

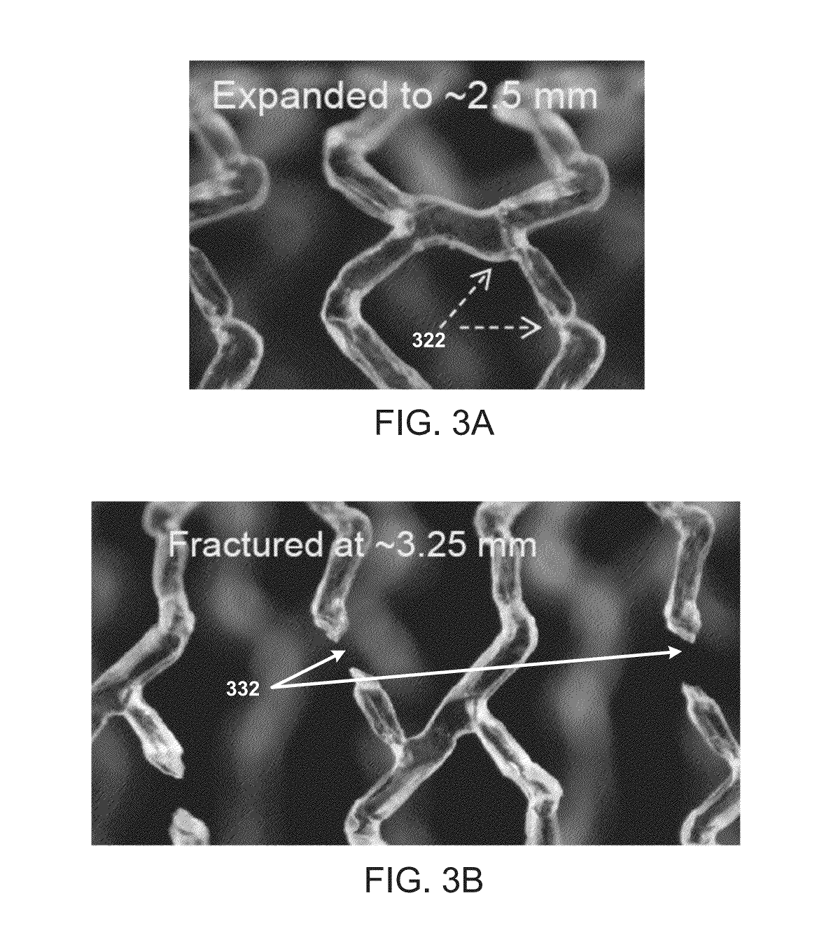

The pattern of stents provided herein can allow for radial expansion, radial compression and longitudinal flexure. The pattern includes struts that are straight or relatively straight, and bending elements. Bending elements bend inward when a stent is crimped to allow radial compression of the stent in preparation for delivery through an anatomical lumen. Bending elements can also bend outward when a stent is deployed to allow for radial expansion of the stent within the anatomical lumen. After deployment, stents provided herein can be subjected to static and cyclic compressive loads from the vessel walls. Thus, bending elements may deform during use. These bending elements, primarily at the peaks and at connector intersections, can deform during crimping and expansion. FIGS. 2, 3A, and 3B show how crimping and expansion can result in thinning and fracturing of bioerodible polymer at the peaks and connector intersections. As shown in FIG. 2, peak 212 and connector intersection 214 each show an uneven plastic deformation in a crimped stent. As shown in FIG. 3A, a stent expanded to 2.5 mm shows thinning 322 at peaks and at connector intersections. As shown in FIG. 3B, the stent of FIG. 3B expanded to 3.25 mm can fracture at peaks 332. Accordingly, devices provided herein can have a bioerodible polymer crosslinked or chain extended at one or more bending elements (e.g., at one or more peaks and/or one or more connector intersections) to provide a material with increased resiliency along these bending elements.

### Fabrication and Use

A polymeric implantable medical device, such as a stent, can be fabricated using any suitable set of processing steps. In some cases, an implantable medical device provided herein can be formed using the following steps:

(1) forming a polymeric construct, such a tube, by using a melt processing step or method, such as an extrusion step, of a photo active functionalized bioerodible polymer provided herein;

(2) radially and axially deforming the formed tube;

(3) cutting (e.g., laser machining) fenestrations into the deformed tube to create a tubular network including a plurality of bands and connectors;

(4) selectively laser photo-crosslinking or chain extending of one or more peaks and/or connector intersections, or photo-crosslinking or chain extending the entire stent

(5) optionally applying of a therapeutic coating on a tubular network;

(6) crimping the tubular network over a catheter balloon; and

(7) sterilizing;

In some cases, one or more of these process steps can be excluded or rearranged. For example, in some cases, select portions of a tubular member can be cross-linked prior to cutting fenestrations. In some cases, a polymeric construct can be obtained from a third party.

A polymer tube including a photo active functionalized bioerodible polymer provided herein can be formed through melt processing methods, such as extrusion and injection molding. In extrusion, a polymer melt is conveyed through an extruder barrel to an exit port. The polymer is fed to an extruder barrel near its proximal end in a solid form, for example, as a pellet from a hopper. The polymer in the extruder barrel is heated to temperatures near or above the melting temperature (Tₘ) of the polymer and exposed to shear forces and pressures that are generally far above ambient pressure. Since the viscosity decreases with temperature, the temperature is at a level that allows a desired flow rate of polymer through the extruder. Exemplary processing temperatures in an extruder can be at or about the Tₘ of the polymer, up to 10° C. above, 10-30° C. above, 30-50° C. above, or more than 50° C. above the Tₘ of the polymer. In some cases, the photo active functionalized bioerodible polymer provided herein can be thermally stable during a melt processing step such that the elevated temperatures do not cause the photo active group to degrade. For example, a PLLA having terminal cinnamate groups can be extruded at a temperature of at least about 180° C., 180-210° C., 210-230° C., or greater than 230° C. The polymer melt exits a distal end of the extruder barrel into a die. The die imparts a cylindrical shape to the polymer melt exiting the die, which is cooled to form a tube.

Radial and axial expansion of the polymer tube may be performed to increase the radial strength and axial strength of the tube, prior to cutting fenestrations to form a tubular network. Generally, deformation of a polymer construct can result in an increase in strength in the deformed construct along the direction of deformation. Therefore, radial and axial expansion or deformation can result in an increase in radial and axial strength of a polymer tube. The increase in strength is believed to arise from molecular orientation and crystallization induced along the direction of deformation, which can increase the strength and modulus along the direction of deformation.

Laser cutting or machining can be used to cut a stent pattern in a polymer tube. Laser cutting can result in chemical degradation in all or a part of the tube due to heat and radiation exposure from the laser beam. In particular, chemical degradation can be most significant in selected or localized regions called a heat affected zone (HAZ). A HAZ typically refers to sections of a target substrate that are adjacent to sections of removed substrate material that were not removed and exposed to energy from the laser beam, either directly or indirectly. Direct exposure produces a HAZ when a section of the beam with an intensity comes in contact with sections of the target substrate, but the intensity is not great enough to remove substrate material. A substrate can also be exposed to energy indirectly due to thermal conduction and radiation scattering. The increased temperature in a HAZ can lead to chemical degradation of the polymer. In some cases, photo active functionalized bioerodible polymer provided herein can be thermally stable during laser cutting or machining such that elevated temperatures associated with the HAZ do not cause the photo active group to degrade. In some cases, a photo active group used in bioerodible polymers provided herein can be triggered to degrade at select wavelengths since a laser used to cut fenestrations can use different wavelengths.

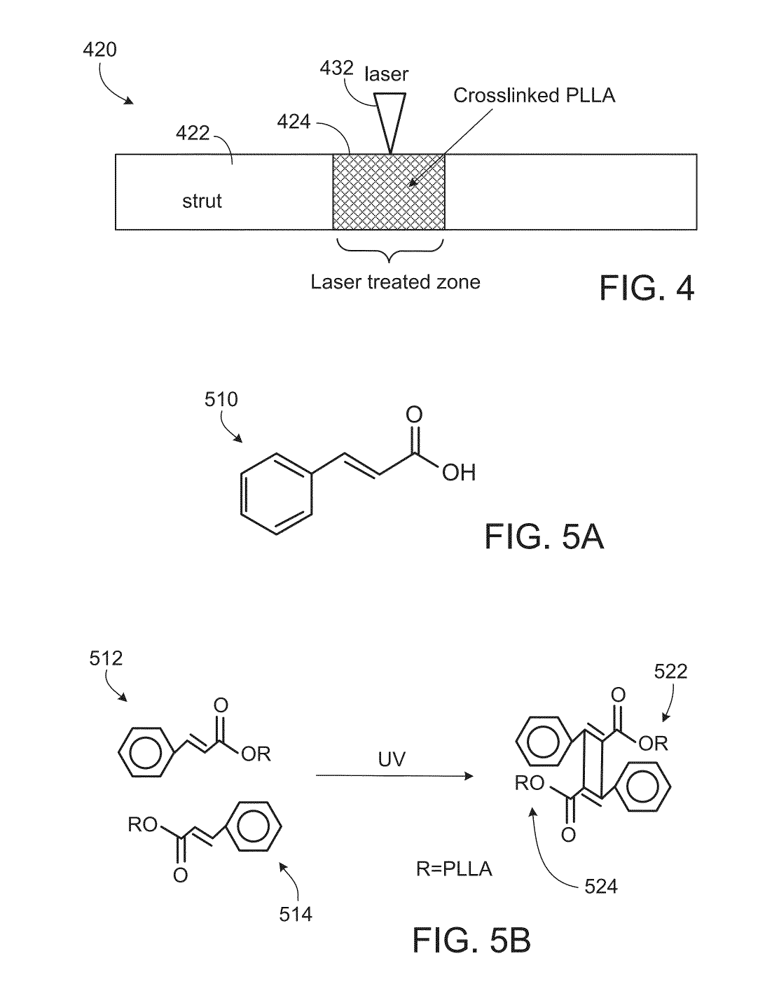

Selective crosslinking or chain extending of select areas of a tubular network can be achieved by directing/focusing light towards those select areas. The light can have a predetermined wavelength. The predetermined wavelength can be based on the photo active functional group. FIG. 4 depicts how a laser 432 can be focused onto a select portion 424 of a stent strut 420 to crosslink or chain extend the photo active functionalized bioerodible polymer in the section portion 424 without crosslinking or chain extending surrounding portions 422 of the stent strut 420. In some cases, laser 432 can be an excimer laser. In some cases, the section portion 324 can be a peak in a band. In some cases, the select portion 424 can be a connector intersection. In some cases, the photo active functional group can be a cinnamate functional group and laser 432 can provide UV light having a wavelength of 365 nm. Cinnamates can undergo “crosslinking” (cycloaddition) at 365 nm. In some cases, laser 432 can supply UV light to a target region or to the stent globally for less than 1 minute, for between 0.01 seconds to 30 seconds, for between 0.1 seconds and 10 seconds, or between 0.5 seconds and 5 seconds. Representative examples of lasers that may be used include, but are not limited to, excimer, carbon dioxide, and YAG lasers.

A coating step may optionally provide a medical device provided herein with a therapeutic agent release layer. The coating may be polymeric or non-polymeric and may include an active agent. A polymeric coating can be formed by applying a coating composition containing a polymer dissolved in a solvent. An active agent can be mixed or dispersed in the solvent as well. The coating composition is generally applied at ambient temperature (20-30° C.). The coating is formed when the solvent is removed using a drying step that can include the application of heat to the tubular network. The drying step can include blowing a heated gas on the tubular network, for example, for 10 to 45 seconds, at 35-45° C. or greater than 45° C. The application and drying steps can be repeated several times to achieve a desired mass of polymer coating, active agent, or both. When coating application is completed, residual solvent can be removed by heating the coated stent to temperatures of 40-65° C., or greater than 65° C. For example, the coated stent can be placed in a vacuum oven for a time of 30-180 minutes. Photo active functionalized bioerodible polymers provided herein can be adapted to be resistant to degradation of the photo active functional group during exposure to the elevated temperatures during the drying processes.

Medical devices provided herein can be mounted on a support element, such as a catheter balloon, by crimping the tubular network over the support element. In some cases, a stent can be heated to a temperature above ambient during the crimping process. The crimping of a polymeric stent can be facilitated and mechanical behavior of the stent after crimping can be improved by such heating. For example, heating a stent during crimping can reduce or eliminate recoil of a crimped stent prior to delivery in a lumen. In some cases, a stent can be crimped at a temperature in a range up to the Tg or above the Tg of the scaffolding polymer, for example, between −5% to 5% of the Tg of the polymer of the scaffolding polymer, or greater than 5% above the Tg of the scaffolding polymer. For example, for a PLLA stent, crimping may be performed at a temperature range of 30-70° C., or a temperature greater than 70° C. for a duration ranging from about 60 seconds to about 5 minutes. Photo active functionalized bioerodible polymers provided herein can be adapted to be resistant to degradation of the photo active functional group during exposure to the elevated temperatures during the crimping processes.

Sterilization of a medical device provided herein can be accomplished after crimping and packaging the crimped tubular network. Ethylene oxide sterilization, or irradiation, either gamma irradiation or electron beam irradiation (e-beam irradiation), are typically used for terminal sterilization of medical devices. For ethylene oxide sterilization, the medical device is exposed to liquid or gas ethylene oxide that sterilizes through an alkylation reaction that prevents organisms from reproducing. Ethylene oxide penetrates the device, and then the device is aerated to assure very low residual levels of ethylene oxide because it is highly toxic. The ethylene oxide sterilization is often performed at elevated temperatures to speed up the process. Photo active functionalized bioerodible polymers provided herein can be adapted to be resistant to degradation of the photo active functional group during exposure to the elevated temperatures during an ethylene oxide sterilization process.

Radiation sterilization can cause chemical degradation to a bioerodible polymer, but photo active functionalized bioerodible polymers provided herein can be resistant to degradation of the photo active functional group when exposed to sterilization irradiation. High-energy radiation, such as electron beams (e-beam) and gamma radiation, tends to produce ionization and excitation in polymer molecules. These energy-rich species undergo dissociation, subtraction, and addition reactions in a sequence leading to chemical degradation. The degradation can occur during, immediately after, or even days, weeks, or months after exposure to radiation, which often results in physical and chemical crosslinking, chain extension or chain scission. Resultant physical changes can include embrittlement, discoloration, odor generation, stiffening, and softening, among others. In particular, the deterioration of the performance of polymers due to e-beam radiation sterilization has been associated with free radical formation during radiation exposure and by reaction with other parts of the polymer chains. The reaction is dependent on e-beam dose and temperature. Additionally, exposure to radiation, such as e-beam, can cause a rise in temperature of an irradiated polymer sample. The rise in temperature is dependent on the level of exposure. In particular, the effect of radiation on mechanical properties becomes more profound as the temperature approaches and surpasses the Tg of the polymer. The deterioration of mechanical properties may result from the effect of the temperature on polymer morphology, but also from increased degradation resulting in a decrease in molecular weight. The degree of chemical degradation can be reduced by irradiating the stent before, during, or after the stent is cooled to a temperature below ambient temperature. As an example, without limitation, a stent can be sterilized while the stent is at a temperature of less than −30° C., −30 ° C. to 0.0° C., or 0.0° C. to 250° C. The sterilization may occur in multiple passes through the electron beam. In some cases, the stent can be at ambient temperature when it is irradiated. Polymer molecular weight may significantly decrease during the processing steps of stent manufacture. Nonetheless, photo active functionalized bioerodible polymers provided herein can be adapted to be resistant to degradation of the photo active functional group during exposure to the gamma irradiation or electron beam irradiation during irradiation sterilization processes.

A non-limiting example of stent fabrication process provided herein is the fabrication of a stent having a stent scaffolding made from cinnamate functionalized PLLA polymer. The stent manufacturing process involves extruding a polymer tube, radially expanding the polymer tube, laser cutting a stent pattern into the tube to form a stent, directing UV light having a wave length of 365 nm towards the peaks and connector intersections, crimping the stent onto a balloon catheter, and sterilizing the crimped stent with e-beam radiation.

FIG. 5A illustrates the structure of cinnamic acid 510, which is an organic compound with the formula C₆H₅CHCHCO₂H. It is a white crystalline compound that is slightly soluble in water. Classified as an unsaturated carboxylic acid, it occurs naturally in a number of plants. It exists as both a cis and a trans isomer, although the latter is more common. In some cases, cinnamic acid can be formed using the Perkin reaction, which entails the base-catalysed condensation of acetic anhydride and benzaldehyde. The reaction is known as the Claisen condensation. Cinnamic acid can also be prepared from cinnamaldehyde and benzal chloride. Cinnamic acid 510 can be linked to a terminal end of a bioerodible polymer having a —OH end group using an esterification reaction.

FIG. 5B depicts how terminal cinnamate functional groups 512 and 514 can undergo 2+2 cycloaddition when exposed to UV light resulting in a chain extension or the crosslinking of PLLA (522 and 524)

A number of embodiments of the invention have been described. Nevertheless, it will be understood that various modifications may be made without departing from the spirit and scope of the invention. Accordingly, other embodiments are within the scope of the following claims.

What is claimed is:

1. A medical device comprising:
a tubular network of struts comprising a bioerodible polymer, the bioerodible polymer comprising a plurality of polymer chains functionalized with a photo active group the tubular network comprising a plurality of bands and a plurality of connector struts, each band including a plurality of peaks between adjacent band struts and being connected to one or more adjacent bands by at least two connector struts at a plurality of connector intersections, the tubular network comprising greater crosslinking or chain extension between polymer chains of the plurality of polymer chains in one or more peaks, one or more connector intersections, or a combination thereof as compared to one or more midsections of adjacent struts.

2. The medical device of claim 1, wherein the photo active group is a cinnamate functional group.

3. The medical device of claim 2, wherein the plurality of the polymer chains each comprise two or more terminal cinnamate functional groups.

4. The medical device of claim 1, wherein the plurality of polymer chains are heat stable such that exposure to a temperature of 215° C. for 20 minutes does not cause more than 5% of the plurality of polymer chains to crosslink or chain extend.

5. The medical device of claim 1, wherein the bioerodible polymer comprises a polymer selected from the group consisting of PLGA, PDLA, PLLA, PCL, PHBV, POE, PEO/PBTP, one or more polyamides, one or more polyanhides, and a combination thereof.

6. The medical device of claim 5, wherein the bioerodible polymer consists essentially of PLLA functionalized with cinnamate functional groups.

7. The medical device of claim 1, wherein the bioerodible polymer has a number molecular weight of at least 30,000 and a Tg of at least 40° C.

8. The medical device of claim 1, wherein each peak comprises crosslinked or chain extended bioerodible polymer and each band strut between the peaks comprises bioerodible polymer that is less crosslinked or chain extended than the peaks.

9. The medical device of claim 1, wherein the medical device is a stent.

10. A method of forming a medical device comprising focusing light onto one or more predetermined locations of a tubular network, the tubular network comprising a bioerodible polymer, the bioerodible polymer comprising a plurality of polymer chains functionalized with a photo active group, the tubular network comprising a plurality of bands and a plurality of connector struts, each band including a plurality of peaks between adjacent band struts and being connected to one or more adjacent bands by at least two connector struts at a plurality of connector intersections, the light having a wavelength adapted to trigger the crosslinking or chain extension of the bioerodible polymer in the one or more predetermined locations.

11. The method of claim 10, wherein the light is UV light have a predetermined wavelength.

12. The method of claim 10, further comprising forming the tubular network by melt extruding the bioerodible polymer into a tube and cutting fenestrations in the tube to define the bands and the connectors, wherein the melt extruding of the bioerodible polymer does not trigger the decomposition of the photo active group.

13. The method of claim 10, further comprising sterilizing the tubular network with e-beam radiation, wherein the e-beam radiation does not trigger the decomposition of the photo active group.

14. The method of claim 10, further comprising functionalizing a bioerodible polymer with one or more functional groups to form the bioerodible polymer.

15. The method of claim 10, wherein the photo active group is a cinnamate functional group.

16. The method of claim 10, wherein the plurality of the polymer chains each comprise two or more terminal cinnamate functional groups.

17. A stent comprising a tubular network of struts comprising a PLLA chains functionalized with one or more cinnamate functional groups.

18. The stent of claim 17, wherein a plurality of PLLA chains each comprise two terminal cinnamate functional groups.

19. The stent of claim 17, wherein the PLLA chains are heat stable such that exposure to a temperature of 215° C. for 20 minutes does not cause more than 5% of the plurality of polymer chains to crosslink or chain extend or chain extend.

20. The stent of claim 17, wherein the functionalized PLLA chains have a number molecular weight of at least 30,000 and a Tg of at least 40° C.
